(19) United States
(12) Patent Application Publication
(10) Pub. No.: US 2011/0166442 A1
(43) Pub. Date: Jul. 7, 2011

(54) SYSTEM FOR OPTICALLY DETECTING POSITION OF AN INDWELLING CATHETER

(75) Inventor: Armen P. Sarvazyan, Lambertville, NJ (US)

(73) Assignee: ARTANN LABORATORIES, INC., Lambertville, NJ (US)

(21) Appl. No.: 12/683,652

(22) Filed: Jan. 7, 2010

(51) Int. Cl. A61B 6/00

(52) U.S. Cl. 600/424

(57) ABSTRACT

The present invention relates generally a device for locating an indwelling catheter relative to its initial location. The system of the invention is based on emitting light from an optical probe placed on the patient to an optical marker on the tip of the catheter. The reflected light from the optical marker is then detected by the optical probe and the reading is recorded to memory as the reference measurement. The position of the optical probe on the patient is marked so that future measurements are taken from the same location. These future measurements will be compared to the reference measurement and from this comparison the displacement of the tip of the catheter is found and can be corrected. This system is fast, non-invasive, radiation free, and accurate to within 2-3 mm.

### BACKGROUND OF THE INVENTION

The present invention relates to a quick, non-invasive, radiation-free device to determine a position of indwelling catheters within the human or animal body. The term catheter as used throughout this description refers to any type of invasive surgical tool, used for insertion into a human or animal body for the purpose of providing remote access to a part of the body for performing some type of investigative and/or therapeutic medical procedure. Examples of such tools include various catheters, tubes, endotracheal tubes, cannulaes, probes etc.

With the increasing use of minimally invasive surgical techniques in medical diagnosis and therapy, there is a need for new methods of remotely locating and tracking catheters or other medical instruments inside a human or animal body. Currently, X-ray imaging is the standard catheter tracking technique. However, excessive exposure to X-ray radiation by both the patient and clinician can be harmful. Thus, alternative catheter tracking methods are desirable.

Several such methods have been published including some which employ magnetic field measurements and others using ultrasonic or optical measurements. U.S. Pat. No. 6,349,720 for example describes a device indicating a position of a catheter with varying sounds. Such a system requires a medical caregiver to listen and determine if the sound has been heard from both sides of the chest cavity equally or if the sound was heard from the stomach. This method requires some subjectivity which limits its ultimate effectiveness.

One example of a magnetic catheter tracking system is disclosed in the U.S. Pat. No. 6,783,536—it shows a catheter-stiffening insert wire incorporating a distal magnet, which is traced from outside the body by a system with magnetic sensors. An important limitation of this device is the need to gain access inside the catheter for its proper function. A general limitation of magnetic tracking systems is a risk of artifacts from surrounding large metal objects such as a rail of a patient's bed or other medical equipment.

Another device is shown in the U.S. Pat. No. 4,567,882—it provides an insert into a catheter containing an optical fiber transmitting light along the length of the tube. In order to align or monitor the position of the tube, a light source is connected to the external end of the tube causing the internal tip of the tube to glow within the patient's body. This device fails to specifically determine if the tube is properly positioned, again adding subjectivity to the procedure by requiring the medical caregiver to determine if the glowing portion appears to be in the correct location.

Eliminating the need to use X-rays for monitoring catheter position presents a number of important clinical and financial benefits:
a. Reduction of equipment costs associated with lower utilization of X-ray machines;
b. Reduction of labor costs and time. Instead of a substantial time needed to take an X-ray by a highly trained technician and time for the radiologist to provide a reading (total average 20 minutes), catheter position verification testing could be performed by a nurse within seconds;
c. Increased safety due to an ability to check catheter position more frequently and without the use of hazardous radiation.

The need exists therefore for a catheter position detection device that allows a medical caregiver to objectively and quickly determine if the catheter is properly positioned. This system ideally will be quick, non-intrusive, radiation-free and can be used by one caregiver at the patient's bedside.

### SUMMARY OF THE INVENTION

Accordingly, it is an object of the present invention to overcome these and other drawbacks of the prior art by providing a novel optical detection device for locating and tracking position of an indwelling catheter inside a body.

It is another object of the present invention to provide a radiation-free, non-intrusive catheter-locating system adapted for use by a single medical caregiver at the patient's bedside.

It is a further object of the present invention to provide an optical catheter location detector allowing tracking of a catheter along a predetermined line.

It is yet another object of the invention to provide a catheter locating device allowing locating a catheter in a two-dimensional plane.

In accordance with this invention, there is provided an improved way to locate a catheter though a combination of an optical marker placed on the tip of the catheter and an optical probe consisting of at least one light emitter and two light detectors. The system requires the catheter's tip to have an optical marker imbedded therein or attached thereto before it is inserted and positioned inside the patient at the appropriate location. The optical marker could be an optical reflector or a stripe made with conventional or fluorescent dye, either one is preferably imbedded in the catheter. After the catheter is initially placed at the desired location, an optical probe with at least one light source is placed on the patient's body above the estimated location of the catheter tip. Light passes through the patient's skin towards the optical marker and is then reflected back to the probe's light detectors. The initial reflected signal is measured and stored in the probe's memory as a reference signal. In order to return the optical probe to this position later, the skin is marked at the initial location of the optical probe. The skin reference indicia will remain on the patient for the duration of the monitoring period. Future measurements will record the light strength to compare it to the reference signal in order to determine the current location of the catheter's tip relative to the initial correct location. The catheter position can be adjusted until the current signal matches the reference signal. Importantly, although the presence of an interposing soft tissue tens of millimeters thick significantly attenuates the signals, it does not affect the distance-related decay. Comparing the signal strength as measured by at least two light detectors positioned on both sides of the light emitter allows accurate detection of the tip position. In the steep part of the signal/distance curve, the catheter position can be estimated with the accuracy of 2-3 mm.

In additional embodiments of the invention, the optical probe consists of a single light emitter and a linear array of light detectors, and a single light emitter with two pairs of light detectors oriented transversely to each other. Any of these embodiments could also be supplemented with a narrowband optical filter limiting the wavelength range of light directed through the tissue. Another useful supplemental element is an opaque shield covering surrounding areas of skin in order to enhance position detection of the reflected light by blocking ambient light. In a further alternative, the light emitter is envisioned to generate an amplitude-modulated light beam formed by short-term light flashes, rectangular light pulses, or harmonic light oscillations.

### BRIEF DESCRIPTION OF THE DRAWINGS

A more complete appreciation of the subject matter of the present invention and the various advantages thereof can be realized by reference to the following detailed description in which reference is made to the accompanying drawings in which:

FIG. 1 is a general view of an optical probe of the invention positioned on a patient's body above an optical marker on a tip of an indwelling catheter.

FIG. 2 is a schematic view of the probe with a light emitter and two light detectors positioned over the optical marker on the tip of the catheter according to the first embodiment of the invention.

FIG. 3 is a graphical representation of the optical position detection principle of the invention.

FIG. 4 is a schematic view of a light emitter and a linear array of light detectors positioned over the optical marker on the tip of the catheter according to the second embodiment of the invention.

FIG. 5 is a schematic view of a light emitter and transversely oriented pairs of light detectors positioned over the optical marker on the tip of catheter according to the third embodiment of the invention.

FIG. 6 is a graphical representation of the detection of a two dimensional displacement of the catheter's tip.

FIG. 7 is a schematic view of a light emitter and two light detectors equipped with an optical filter and positioned over the optical marker on the tip of the catheter.

FIG. 8 is a graphical representation of the optical filtering of the absorption and emission wavelengths of the fluorescent dye.

FIG. 9 is a schematic view of the optical detection of an amplitude-modulated light beam.

FIG. 10 is a schematic view of the optical probe with a deployed opaque shield over the optical marker on the tip of the catheter.

FIG. 11 is a block-diagram of the probe of the invention.

### DETAILED DESCRIPTION OF PREFERRED EMBODIMENTS OF THE INVENTION

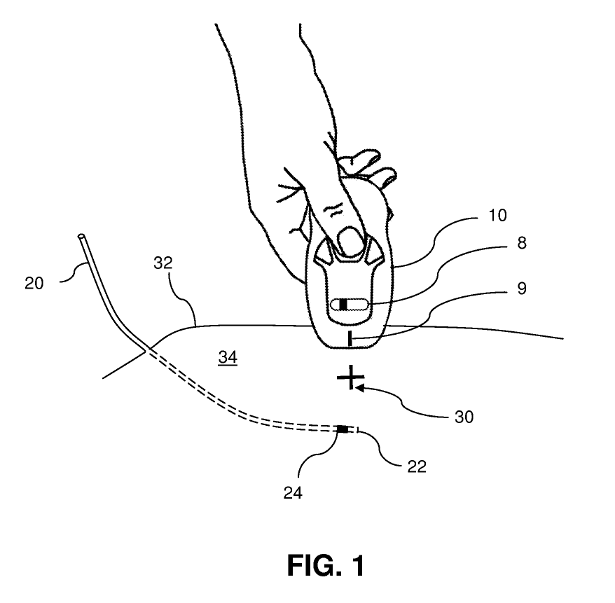

FIG. 1 presents a general view of an optical probe 10 positioned on a patient's body 34 over the tip 22 of a catheter 20. The tip 22 has an optical marker 24 on or close to its outer surface. The marker 24 can be an optical reflector such as a painted strip or a dot on a catheter made with a conventional or fluorescent dye. The optical marker 24 is preferably permanently built into the catheter (such as an imbedded band or painted strip) or optionally constitutes an add-on component to be attached to an existing device before insertion. The optical probe 10 is placed on the skin 32 of the patient after the catheter 20 has been inserted to the correct location inside the body. The probe contains skin reference stripe 9. The skin is marked with the skin reference 30 next to stripe 9 so as to allow returning the probe to this location for later measurements. In one embodiment of the invention, the light measurements taken directly after positioning the catheter tip 22 at the correct location are recorded by the optical probe 10 and are stored as a reference signal. During the first placement, the catheter position is confirmed by X-ray or other means.

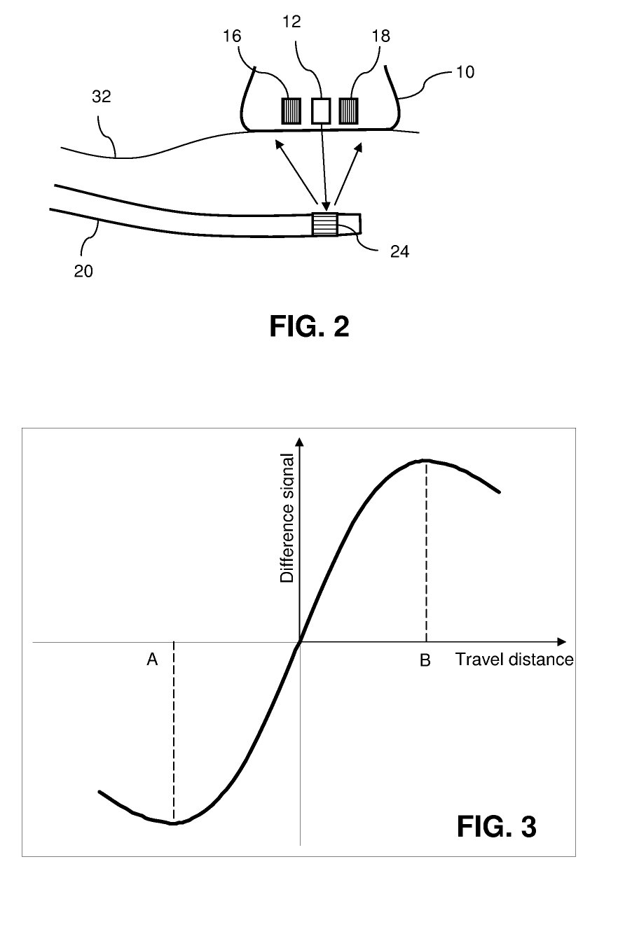

FIG. 2 is a schematic view of the first embodiment of the present invention in which a probe 10 includes a light emitter 12 and a pair of light detectors 16 and 18 positioned on both sides of the emitter 12 along a first axis aligned with the projected travel path of the catheter, preferably at an equal distance therefrom. The probe 10 is shown positioned over the optical marker 24 at the tip of a catheter 20. The light emitter 12 may be a light-emitting diode (LED) while the light detectors 16 and 18 may be photodiodes. Preferably, a high-speed high-power infra-red LED is used as a light emitter 12 while an integrated photodiode and amplifier is used as a light detector 16 and 18. To ensure the highest tissue penetration and the least absorption when passing through tissue, the range of wavelengths for the light emitter 12 is preferably selected to be from about 650 nm to about 900 nm. Shorter wavelengths may cause increased absorption by hemoglobin in blood while longer wavelengths may be absorbed by water [Nam Jung Kim; Hyun Soo Lim. Measurements of absorption coefficients within biological tissue in vitro. Engineering in Medicine and Biology Society, 1998. Proceedings of the 20th Annual International Conference of the IEEE, Volume 6, 2960-2962.] The base of the probe 10 is preferably oriented in parallel with a projected travel path of the catheter 20 inside the body 32 and consequently the light emitter 12 and light detectors 16 and 18 are aligned along the expected movement trajectory of the optical marker 24.

FIG. 3 graphically illustrates a general principle behind the optical position detection of the present invention. The vertical axis shows a difference between light signal as measured by the two light detectors 16 and 18. The horizontal axis shows the travel distance of the catheter tip under the probe. Starting from the left, both signals are weak and the difference between them is negligible. As the catheter moves closer to detector 16, it measures increased signal strength while detector 18 is still far away and measures a weak signal. Subtracting detector 16 from detector 18 gives a strong negative value which has its negative peak (point A on the curve) when the tip is located at an equal distance between detector 16 and emitter 12. As the catheter is moved further to the right, detector 16 measures lower signal strength while detector 18 measures higher signal strength so the curve starts to move higher. It reaches zero when both detector measure the same signal strength—the tip at this point is located under the light emitter 12. Moving the tip further to the right causes continuous decrease in signal strength measured by detector 16 and increase in signal strength measured by detector 18. The curve reaches its positive peak (point B on the curve) when the tip is located at an equal distance between the light emitter 12 and the light detector 18 so the light travel path is minimal. Continuous movement of the tip further to the right causes decrease in both signals. Depending on a specific application, the distance between detectors 16 and 18 is selected to maximize accuracy of the probe as it is highly sensitive to the tip position between points A and B on the curve.

The main advantage of having two detectors 16 and 18 on both sides of the emitter 12 comes from the ability to detect the catheter position by comparing a signal measured by one detector to that measured by another. Relative measurement of signals eliminates errors caused by changing light and tissue conditions. These changing conditions equally affect both detectors and therefore are eliminated when one signal is subtracted from the other.

The use of the probe 10 starts with positioning the catheter tip at the anatomically appropriate locations and verifying this position by other means such as an X-ray. The probe is then placed on the skin and a mark is made to be able to return the probe to the same place later. Initial signal is recorded and stored in the probe as a reference signal. Subsequent verification of location is done by positioning the probe at the same place on the skin, turning it on and instructing the caregiver to move the catheter a little back and forth. Such movement will be recognized by the probe as a change in signal so that subtraction of signal from one detector from the signal recorded by the other detector will produce an accurate position verification input and will cancel out noise associated with changing of ambient light, slight shift in tissue position or perhaps some swelling of the tissue. The probe is adapted to indicate the present position of the catheter and guide the caregiver to move the catheter back to the initial location should any deviation in its position is detected.

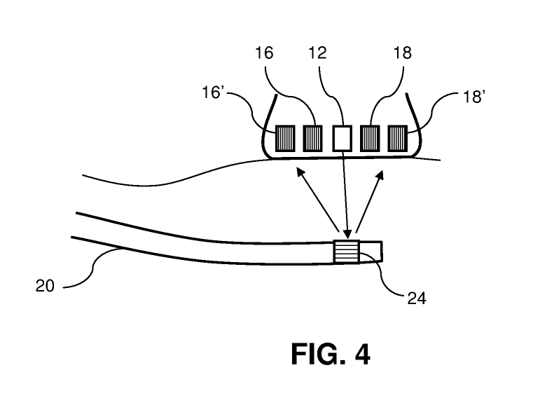

FIG. 4 is a schematic view of the second embodiment of the optical probe 10 with a light emitter 12 and a linear array of light detectors including a first array of at least two light detectors 16 and 16′ on one side of the light emitter 12 and a second array of at least two light detectors 18 and 18′ on the other side of light emitter 12. The light emitter 12 and the light detectors 16, 16′, 18, and 18′ are placed along a probe axis formed as a straight line and oriented along a projected travel path of the catheter tip with the embedded optical marker 24.

There are several advantageous ways to utilize detector arrays of this embodiment. In one way, all detectors are turned on at all times during the catheter position identification process. Having more than one detector allows further reduction in noise and increase in accuracy of position detection. Alternatively, these detectors can be turned on and off at various stages of catheter detection procedure. At first, outside detectors can be turned on to increase the range of detection for the probe as it is more sensitive in the space between detectors. As the probe identifies the moving catheter tip using outside detectors, it turns on inside detectors to increase the accuracy of position detection.

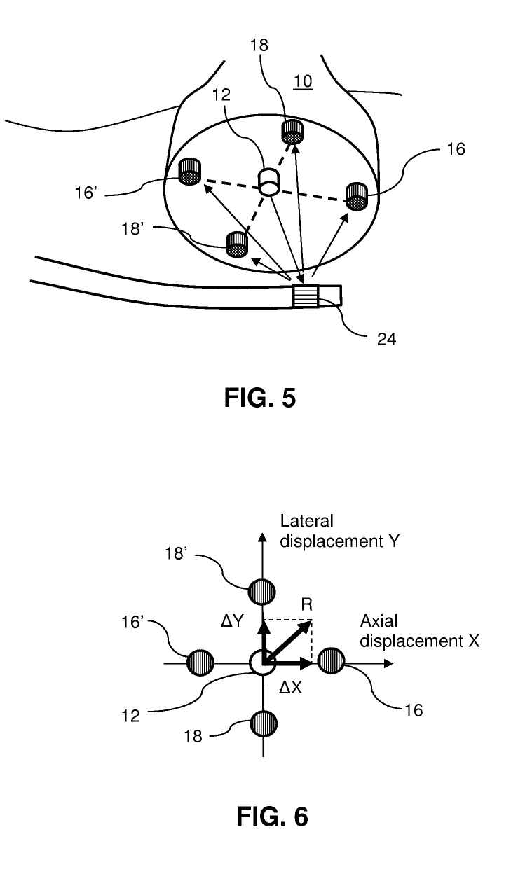

FIG. 5 is a schematic view of the third embodiment of the invention in which the probe includes a light emitter 12 and two transversely oriented pairs of the light detectors 16 and 16′ along a first axis as well as 18 and 18′ along a second axis. The first pair of detectors 16 and 16′ is preferably oriented along the projected travel path of the catheter tip with the optical marker 24, while the second pair of light detectors 18 and 18′ are placed on an axis orientated in the perpendicular direction. Detection of both magnitude and direction of travel of the optical marker 24 is carried out in the axial and lateral directions by comparing the current optical signals with the reference signals.

FIG. 6 shows the principle behind the detection of a two dimensional displacement of the catheter tip as the combination of two vectors corresponding to the axial and lateral displacements which are measured by transversally oriented pairs of light detectors with a light emitter 12 positioned in the center. Each vector is measured individually using the principle described above for the first embodiment of the invention.

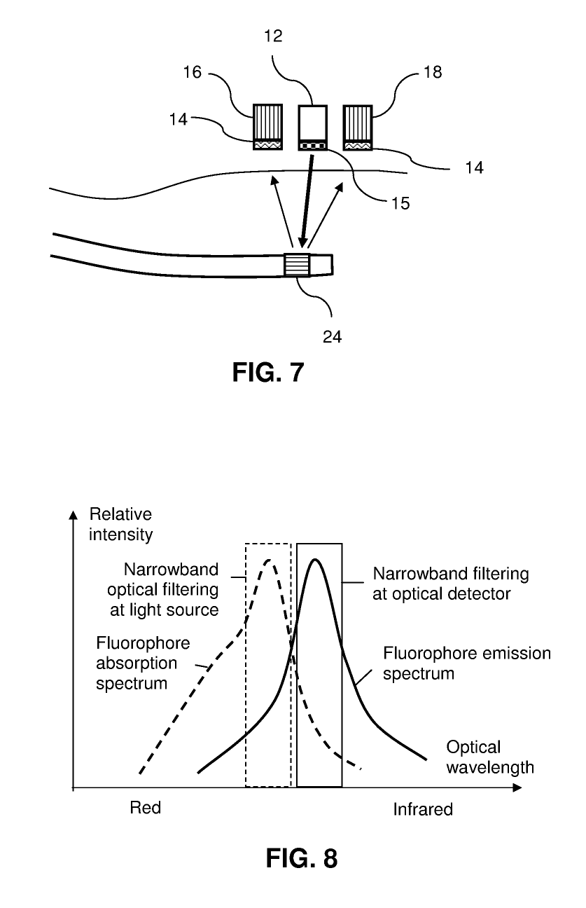

FIG. 7 presents a schematic view of the probe adapted to work with the catheter having an optical marker made using a fluorescent dye. Fluorescent marker is made with a fluorophore having a known absorption spectrum and emission spectrum as shown in FIG. 8. The light emitter 12 and a pair of light detectors 16 and 18 are equipped with corresponding narrowband optical filters 15 and 14. The filter 15 at the light emitter 12 allows light transmission of the incident light beam to be at the wavelength corresponding to the peak of absorption spectra of the applied fluorescent dye on the optical marker 24. The filters 14 at the light emitters 16 and 18 are selected to allow for light transmission at the wavelength corresponding to the peak of emission spectrum of the fluorophore so as to block all other ambient light. Spectral parameters of light emitter 12 and light detectors 16 and 18 are therefore matched to the absorption and emission spectra of the fluorophore.

FIG. 8 displays in more detail the principle of optical filtering when the optical marker 24 is a fluorescent dye. To penetrate the layer of biological tissues, red or near infrared light is emitted from the light emitter 12. The light passes through a filter 15 which allows only the narrowband of peak absorption wavelengths of fluorescence to pass through. This light is absorbed by the fluorescence of the marker 24 and emitted light is produced. Another filter 14 at each light detector 16 and 18 blocks all wavelengths outside the peak signal of the emitted light. This concept allows an increase of accuracy of position detection for the probe of the present invention.

The following specific components may be used to design the probe according to this embodiment of the invention: laser SDL-650-LM-50 (Shanghai Dream Lasers Techonology Co., Ltd, China) as a light source 12; light sensor TSL257 made by TAOS Inc. (USA) as light detectors 16 and 18; fluorophore Alexa Fluor 660 by Invitrogen Corp. (USA) as a fluorescent dye; and optical filters FF01-655/40-25 for light detectors 16 and 18 and FF01-716/40-25 for light emitter 12 made by Semrock Inc. (USA).

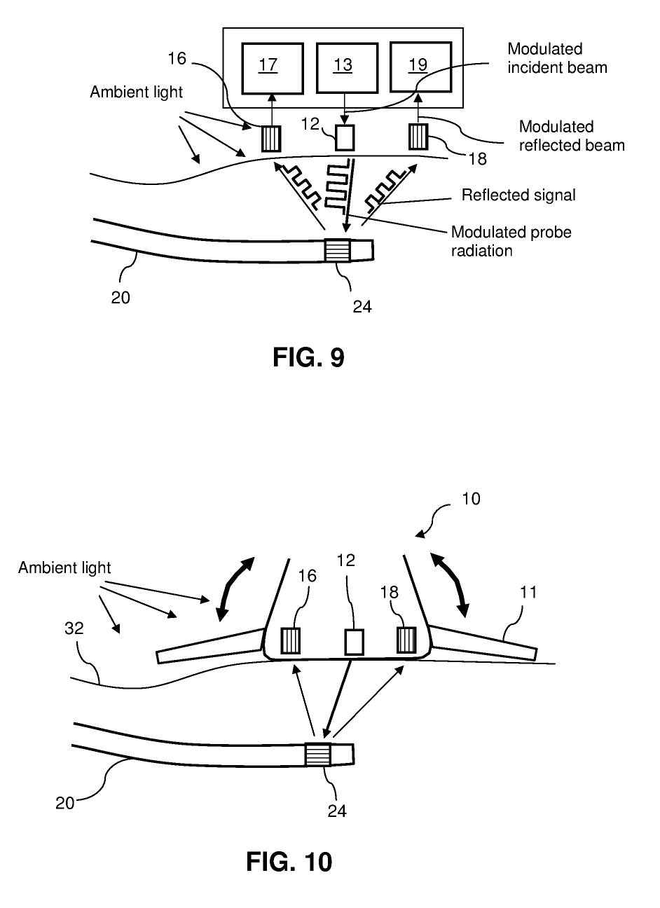

FIG. 9 illustrates another useful improvement raising the accuracy of tip detection for the detection of reflected modulated light from the optical marker 24 on the catheter 20. In order to filter out ambient light, the light emitter 12 is controlled by a radio-frequency modulation circuit 13 that generates an amplitude-modulated light beam pattern formed for example by one of the following type of modulations: short-term light flashes, rectangular pulses or harmonic oscillations. The reflected beam from the optical marker 2 also has the same amplitude-modulated characteristics so that it can be separated from the ambient light by demodulation processing algorithms deployed by programmable logical demodulation circuits 17 and 19 of the respective light detectors 16 and 18.

FIG. 10 presents a schematic view of the optical probe 10 equipped with an opaque shield 11 which protects the area of interest from ambient light. The opaque shield 11 can be initially folded away and hidden from view. After the medical caregiver positions the probe 10 on the marked location of the patient's body, the shield 11 can be extended and placed on a surrounding portion of the skin 32 to block the ambient light from the area of interest. The shield may be a simple skirt made of black fabric or can be a foldable rigid component.

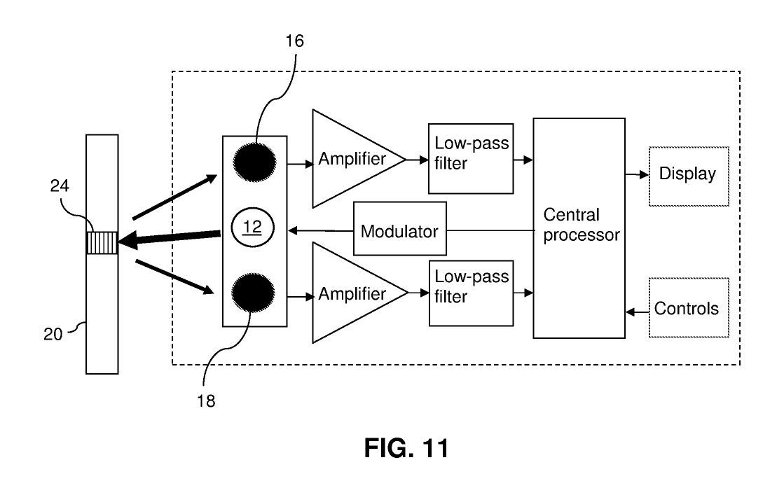

FIG. 11 shows one example of a block-diagram for the probe 10 adapted to work with a combination of two light detectors and a light emitter positioned between thereof as shown for example in FIG. 3. 100 MIPS 8051 core CPU (such as T8051F432 by Silicon Laboratories Inc., Sunnyvale, Calif.) may be used as a central processor of the measurement circuit. It is adapted to manage all optical measurements. 12-bit ADC and DAC may be employed to provide the necessary dynamic range. Central processor is preferably designed to acquire received signals and calculate catheter tip position to then show it on a display such as a built-in linear display 8 as shown in FIG. 1 in which the position of the marker 24 is shown by a corresponding segment of the display 8 being activated.

In use, the optical signal value corresponding to the correct position of the catheter is recorded in the memory of the device by pressing a “set” button on the probe 10. The correctness of catheter position in this initialization may be confirmed with X-ray. For monitoring the subsequent movement of the catheter, the linear position display will show the optical marker 24 displacement so that the nurse may move the catheter back to the appropriate position without additional X-rays.

Although the invention herein has been described with respect to particular embodiments, it is understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A system for detecting position of an indwelling catheter, the system comprising:
an optical marker placed at a distal end of said catheter, and
a probe having a first axis aligned with a projected travel path of said catheter, said probe including at least one light emitter positioned on said first axis and at least a first and a second light detectors, said light detectors placed on said first axis on either side of said light emitter,
whereby said probe is adapted to detect position of optical marker on said catheter using a difference between a light strength signal measured by said first light detector and a light strength signal measured by said second light detector, said light emanating from said light emitter and reflected by said optical marker.

2. The system as in claim 1, wherein said light emitter is adapted to emit light with wavelengths in a range from about 650 nm to about 900 nm.

3. The system as in claim 1, wherein said first light detector is positioned at a distance from said light emitter equal to the distance between said light emitter and said second light detector.

4. The system as in claim 1 further including additional light detectors on said first axis on both sides of said light emitter, said additional detectors forming a first array of detectors and a second array of detectors.

5. The system as in claim 1 further including a second axis placed perpendicular to the first axis through the light emitter, said probe further including a third light detector positioned on said second axis on one side of said light emitter, said probe further including a fourth light detector positioned on said second axis on the other side of said light emitter, whereby said probe is adapted to detect position of said catheter in a two-dimensional plane defined by said four light detectors.

6. The system as in claim 1 wherein said catheter is an endotracheal tube.

7. The system as in claim 1, wherein said optical marker comprises a fluorescent dye.

8. The system as in claim 7, wherein said light emitter further includes a narrowband optical filter to allow passing of light at wavelengths corresponding to absorption peak spectrum of said fluorescent dye.

9. The system as in claim 8, wherein said first and second light detectors are both equipped with a narrowband optical filter allowing passing of light only at wavelengths corresponding to peak emission spectrum of said fluorescent dye.

10. The system as in claim 1, wherein said light emitter is adapted to emit light in a predetermined pattern of amplitude modulation, said probe is adapted to filter out all light outside of said modulations as received by said light detectors.

11. The system as in claim 10, wherein a pattern for said amplitude modulation is selected from a group consisting of short-term light flashes, rectangular light pulses or harmonic light oscillations.

12. The system as in claim 1 further equipped with an opaque shield adapted to block ambient light around said probe.
